(19) United States
(12) Patent Application Publication
(10) Pub. No.: US 2002/0112981 A1
(43) Pub. Date: Aug. 22, 2002

(54) Package and delivery system for bone graft particles

(76) Inventors: Michael B. Cooper, Memphis, TN (US); Stephanie Riley, Gainesville, FL (US); James C. Harber, Memphis, TN (US); Jeff Martin, Germantown, TN (US)

Correspondence Address: FULBRIGHT & JAWORSKI, LLP, 1301 MCKINNEY, SUITE 5100, HOUSTON, TX, 77010-3095, US

(21) Appl. No.: 10/054,523

(22) Filed: Oct. 25, 2001

Related U.S. Application Data
(60) Provisional application No. 60/244,528, filed on Oct. 31, 2000.

(51) Int. Cl. A61B 19/02

(52) U.S. Cl. 206/438; 206/570

(57) ABSTRACT

The present invention is directed to a packaging and delivery system for bone graft particles comprising an outer tray which contains an inner tray comprising a bowl of the particles and also, in some embodiments, a mixing and delivery tool and/or a syringe containing a suspension material for the particles, such as a gel. The inner tray is comprised of an anti-moisture coating and a lid comprised of foil. The mixing and delivery tool preferably has a trowel on one end and a tamping rod on the other end, wherein both features facilitate delivery and compression of the bone graft particles to a bone defect site.

This application claims priority to U.S. Provisional Patent Application Serial No. 60/244,528 filed Oct. 31, 2000.

### FIELD OF THE INVENTION

The present invention relates generally to a packaging and delivery system for particles. More specifically, the invention is directed to a packaging and delivery system for bone graft particles.

### BACKGROUND OF THE INVENTION

Bone defects, such as bone voids, are often treated by grafting of synthetic or natural material into the defect. The industry standard for packaging bone graft substitutes has been to package the substances in glass vials, due to moisture sensitivity issues. A disadvantage of packaging bone graft substances into glass vials is the risk of the glass vial being dropped in the operating room, thereby causing serious debris in the sterile field. It is widely known in the health care field that glass packaging or other glass components is preferably eliminated in an operating room or treatment room whenever possible. An ideal packaging system for a health care provider would allow ease of preparation and delivery of the bone graft product to a patient while also minimizing the amount of packaging to reduce waste.

Other packaging and delivery systems are available in the art. One example of such is the WRIGHT MEDICAL TECHNOLOGY® OSTEOSET® bone graft product, which is supplied in either a glass vial or a gun dispenser. The WRIGHT MEDICAL TECHNOLOGY® ALLOMATRIX® product, to protect from moisture, is contained in two glass vials which are packaged with a plastic mixing bowl and a mixing tool, all of which are contained in a tray covered in a peel pouch. A syringe for delivery of the product is packaged in a separate peel pouch, and both peel pouches are housed in a box. Furthermore, the mixing end of the tool which accompanies the ALLOMATRIX® product is flat, similar to an oar.

The OSTEOTECH® GRAFTON® demineralized bone matrix is contained in a syringe packaged in a foil overpouch which is contained in a peel pouch. COLLAGRAFT® by ZIMMER® is a bone graft matrix made of collagen and a hyroxyapatite/tricalcium phosphate ceramic strip that is contained in two polyethylene terephthalate glycol (PETG) trays.

Thus, what is missing in the art is a packaging system which provides a moisture-proof environment to moisture-sensitive bone graft particles without utilizing glass in the packaging. This invention fulfills this need by providing a glass-free packaging system having a sterile water-impermeable tray, which houses the moisture-sensitive bone graft particles, contained inside an outer tray to maintain sterility.

### BRIEF DESCRIPTION OF THE DRAWINGS

Numerals in the figures correspond with like numerals in the description of the invention herein.

FIG. 1 illustrates an embodiment of the bowl 1 and a bottom view of the bowl cover 2.

FIG. 2 illustrates an embodiment of the bowl 1 and a top view of the cover 2.

FIG. 3 demonstrates an embodiment of an inner tray 22, a mixing and delivery tool 12, a covered bowl 1, and an outer tray 32.

FIG. 4A shows the inner tray 22 housing the mixing and delivery tool 12 and covered bowl 1 inside the outer tray 32.

FIG. 4B shows the inner tray 22 having a lid 20, wherein the lid 20 is partially peeled back.

FIG. 5 shows a bowl 1 containing a plurality of bone graft particles 24.

FIG. 6 shows a bottom view of a preferred embodiment of the mixing and delivery tool 12.

FIG. 7 shows a front view of a preferred embodiment of the mixing and delivery tool 12.

FIG. 8 shows an isoview of a preferred embodiment of the mixing and delivery tool 12.

FIG. 9 shows a left view of a preferred embodiment of the mixing and delivery tool 12.

FIG. 10 shows a right view of a preferred embodiment of the mixing and delivery tool 12.

FIG. 11 shows a top view of a preferred embodiment of the mixing and delivery tool 12.

### SUMMARY OF THE INVENTION

In an object of the present invention, there is a delivery system for a plurality of bone graft particles, comprising a bowl; an inner tray for housing the bowl; an outer tray for housing the inner tray; and the plurality of bone graft particles. In a specific embodiment of the present invention, there is a mixing and delivery tool. In another specific embodiment, the system further comprises a sterile suspension material. In a specific embodiment, the suspension material is housed in a container selected from the group consisting of a syringe, sachet, Applipak®, and peel pouch. In a further specific embodiment, the suspension material is housed in a syringe. In specific embodiments, the bowl contains the plurality of bone graft particles or the bowl and plurality of bone graft particles are housed within the inner tray. In another specific embodiment, the inner tray is impermeable to water.

In an additional specific embodiment, the bowl further comprises a cover selected from the group consisting of a twist-removable cover, a snap-on cover, or a combination thereof. In a specific embodiment, the surface of the cover further comprises a plurality of surface irregularities. In a further specific embodiment, the surface irregularities are selected from the group consisting of bumps, bulges, grooves, channels, furrows, ruts, points, crests and ridges. In an additional specific embodiment, the surface irregularities are bumps. In another specific embodiment, the ratio of the volume of the particles to a volume of the bowl is at least approximately 1:5. In another specific embodiment, the ratio of the volume of the particles to a volume of the bowl is approximately 1:20. In another specific embodiment, the cover comprises a recessed surface, wherein the surface is on a plane parallel with a topmost horizontal plane of the bowl, and wherein when the cover is on the bowl, the plane of the recessed surface lies beneath the topmost horizontal plane of the bowl. In an additional specific embodiment, the cover comprises a plurality of finger tabs along a rim of the cover.

In a further specific embodiment, the delivery and mixing tool comprises a first end having a trowel and a second end having a tamping rod. In an additional specific embodiment, the tool further comprises a surface having a plurality of surface irregularities. In another specific embodiment, the surface irregularities are selected from the group consisting of bumps, bulges, grooves, channels, furrows, ruts, points, crests, and ridges. In a further specific embodiment tool is comprised of a plastic material. In an additional specific embodiment, the plastic material is selected from the group consisting of polycarbonate, polystyrene, polypropylene, cellulose, polyphenylene oxide, and polyphenylene sulfide.

In a specific embodiment, the inner tray is comprised of polyethylene terephthalate glycol (PETG). In another specific embodiment, the surface of the inner tray comprises a coating of chlorotrifluoroethylene. In an additional specific embodiment, the surface of the inner tray comprises an ACLAR® coating. In another specific embodiment, the inner tray further comprises a snap-holding space for the tool. In an additional specific embodiment, the inner tray comprises a lid, wherein the lid comprises a layer selected from the group consisting of foil, polyethylene, paper laminate and a combination thereof. In another specific embodiment, the inner tray comprises a lid, wherein the lid comprises a foil layer, a polyethylene layer, and a paper laminate layer. In a further specific embodiment, the inner tray comprises a chlorotrifluoroethylene coating and a foil lid. In an additional specific embodiment, the inner tray comprises an ACLAR® coating and a foil lid.

In a specific embodiment, the outer tray is polyethylene terephthalate glycol (PETG). In another specific embodiment, the outer tray further comprises a lid, wherein the lid is high-density polyethylene. In another specific embodiment, the outer tray further comprises a lid, wherein the lid is made of TYVEK®. In an additional specific embodiment, the bowl, the particles, the inner tray and the outer tray are housed in a container. In another specific embodiment, the container is selected from the group consisting of a carton, a bag, a box, a pack, a pocket, a peel pouch, a series of trays, and a combination thereof. In an additional specific embodiment, the bowl, the particles, the tool, the inner tray and the outer tray are housed in a container. In another specific embodiment, the container is selected from the group consisting of a carton, a bag, a box, a pack, a pocket, a peel pouch, a series of trays, and a combination thereof. In an additional specific embodiment, the bowl, the particles, the suspension material, the outer tray, and the inner tray are housed in a container. In another specific embodiment, the container is selected from the group consisting of a carton, a bag, a box, a pack, a pocket, a peel pouch, a series of trays, and a combination thereof. In an additional specific embodiment, the bowl, the plurality of particles, the tool, and the inner tray are sterile.

In another object of the present invention, there is a delivery system for a plurality of particles, comprising a bowl containing a plurality of particles; a syringe comprising a sterile suspension material; an inner tray for housing the bowl and the tool; and an outer tray for housing the inner tray. In a specific embodiment, the syringe is a double-barreled syringe.

In another object of the present invention, there is a packaging system for sterile bone graft particles comprising an inner tray; a plurality of bone graft particles housed within the inner tray, the inner tray being impermeable to water such that the bone graft particles remain free of water; and an outer tray for housing the inner tray. In a specific embodiment, the system further comprises a mixing and delivery tool. In an additional specific embodiment, the system further comprises a bowl. In an additional specific embodiment, the system further comprises a sterile suspension material. In a specific embodiment, the suspension material is housed in a container selected from the group consisting of a syringe, sachet, Applipak®, and peel pouch. In another specific embodiment, the suspension material is housed in a syringe. In a further specific embodiment, the bowl contains the plurality of bone graft particles. In an additional specific embodiment, the bowl and the plurality of bone graft particles are housed within the inner tray. In a specific embodiment, the inner tray is impermeable to water.

In another specific embodiment, the bowl further comprises a cover selected from the group consisting of a twist-removable cover, a snap-on cover, or a combination thereof. In a specific embodiment, the surface of the cover further comprises a plurality of surface irregularities. In an additional specific embodiment, the surface irregularities are selected from the group consisting of bumps, bulges, grooves, channels, furrows, ruts, points, crests and ridges. In another specific embodiment, the surface irregularities are bumps. In a specific embodiment, the ratio of the volume of the particles to a volume of the bowl is at least approximately 1:5. In another specific embodiment, the ratio of the volume of the particles to a volume of the bowl is approximately 1:20. In another specific embodiment, the cover comprises a recessed surface, wherein the surface is on a plane parallel with a topmost horizontal plane of the bowl, and wherein when the cover is on the bowl, the plane of the recessed surface lies beneath the topmost horizontal plane of the bowl. In a specific embodiment, the cover comprises a plurality of finger tabs along a rim of the cover. In an additional specific embodiment, the delivery and mixing tool comprises a first end having a trowel and a second end having a tamping rod. In a specific embodiment, the tool further comprises a surface having a plurality of surface irregularities. In a specific embodiment, the surface irregularities are selected from the group consisting of bumps, bulges, grooves, channels, furrows, ruts, points, crests and ridges. In another specific embodiment the tool is comprised of a plastic material. In an additional specific embodiment, the plastic material is selected from the group consisting of polycarbonate, polystyrene, polypropylene, cellulose, polyphenylene oxide and polyphenylene sulfide.

In a further specific embodiment, the inner tray is comprised of polyethylene terephthalate glycol (PETG). In an additional specific embodiment, the surface of the inner tray comprises a chlorofluorotriethylene coating. In a specific embodiment, a surface of the inner tray comprises an ACLAR® coating. In another specific embodiment, the inner tray further comprises a snap-holding space for the tool. In a further specific embodiment, the inner tray comprises a lid, wherein the lid comprises a layer selected from the group consisting of foil, polyethylene, paper laminate and a combination thereof. In an additional specific embodiment, the inner tray comprises a lid, wherein the lid comprises a foil layer, a polyethylene layer, and a paper laminate layer. In another specific embodiment, the inner tray comprises a chlorotrifluoroethylene coating and a foil lid. In a further specific embodiment, the inner tray comprises an ACLAR® coating and a foil lid. In an additional specific embodiment, the inner tray is comprised of PETG and further comprises a snap-holding space for a syringe containing a suspension material, such as a gel. In another specific embodiment, the inner tray is comprised of PETG and further comprises a foam insert for holding a syringe containing a suspension material, such as a gel. In another embodiment, a second inner tray comprised of PETG and having a TYVEK® lid also comprises a syringe, wherein the second inner tray further comprises a snap-holding space for the syringe or a foam insert for the syringe. In another embodiment, the glass syringe lies in a foam insert within an outer tray, wherein the outer tray is comprised of PETG and a TYVEK® lid.

In a specific embodiment, the outer tray is polyethylene terephthalate glycol (PETG). In another specific embodiment, the outer tray further comprises a lid, wherein the lid is high-density polyethylene. In an additional specific embodiment, the outer tray further comprises a lid, wherein the lid is made of TYVEK®. In a specific embodiment, the bowl, the particles, the inner tray and the outer tray are housed in a container. In another specific embodiment, the container is selected from the group consisting of a carton, a bag, a box, a pack, a pocket, a peel pouch, a series of trays, and a combination thereof.

In another embodiment of the present invention, the system further comprises a bowl and a mixing and delivery tool, wherein the bowl, the particles, the tool, the inner tray and the outer tray are housed in a container. In an additional specific embodiment, the container is selected from the group consisting of a carton, a bag, a box, a pack, a pocket, a peel pouch, a series of trays, and a combination thereof. In another specific embodiment, the system further comprises a bowl, a mixing and delivery tool, and a suspension material, wherein the bowl, the particles, the tool, the suspension material, the inner tray and the outer tray are housed in a container. In a specific embodiment the container is selected from the group consisting of a carton, a bag, a box, a pack, a pocket, a peel pouch, a series of trays, and a combination thereof. In another specific embodiment, the bowl, the plurality of particles, the tool and the inner tray are sterile.

Other and further objects, features and advantages would be apparent and eventually more readily understood by reading the following specification and by reference to the accompanying drawings forming a part thereof, or any examples of the presently preferred embodiments of the invention given for the purpose of the disclosure.

### DESCRIPTION OF THE INVENTION

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

As used herein the specification, “a” or “an” may mean one or more. As used herein in the claim(s), when used in conjunction with the word “comprising”, the words “a” or “an” may mean one or more than one. As used herein “another” may mean at least a second or more. As used herein, “any range derivable therein” means a range selected from the numbers described in the specification, and “any integer derivable therein” means any integer between such a range.

I. Definitions

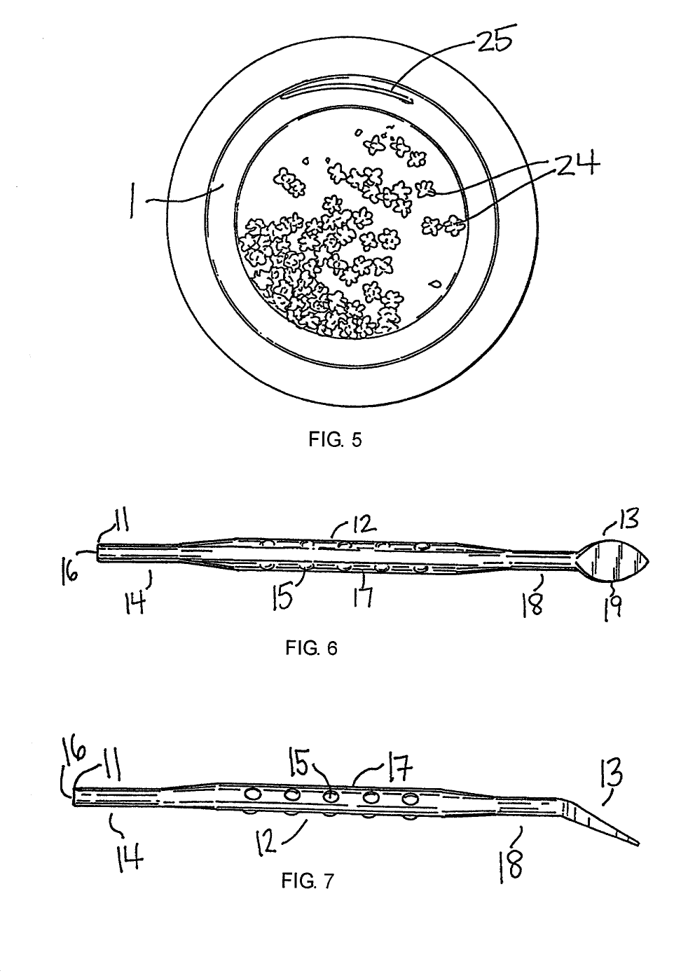

The term “bone graft particles” as used herein refers to shaped particles used to fill a bone defect such as a bone void. In a preferred embodiment, the particles are shaped particles as described in U.S. patent application Ser. No. 09/517,981, incorporated by reference herein in its entirety. In a specific embodiment, the particles are sensitive to moisture. Representative, but not limiting, embodiments of the bone graft particles include 24 of FIG. 5.

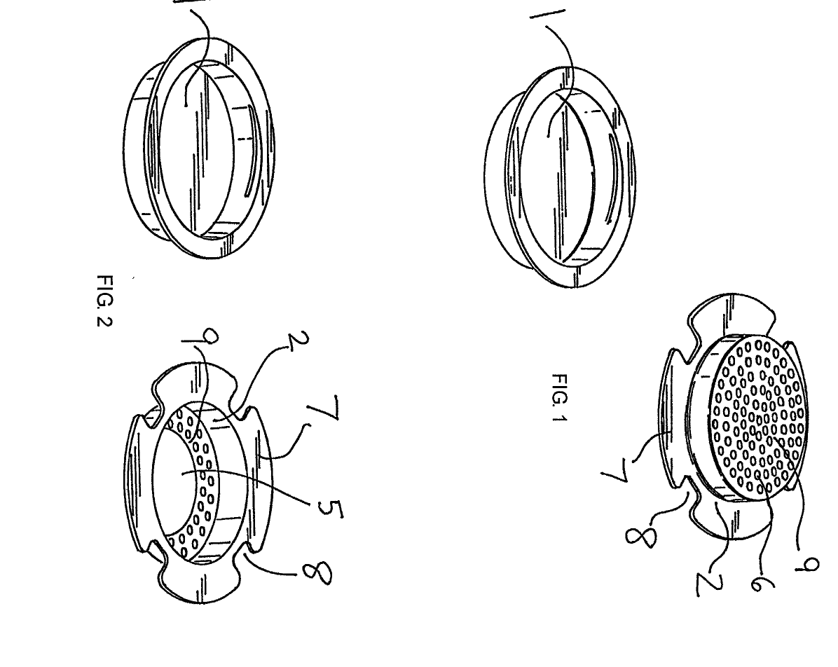

The term “bowl” as used herein is a container for storage and mixing of particles. Representative, but not limiting, embodiments of the bowl include 1 of FIG. 1, FIG. 2 and FIG. 5.

The term “coating” as used herein is defined as a surface covering.

The term “container” as used herein is defined as an apparatus or receptacle for the packaging, shipment, and/or delivery of at least one system component.

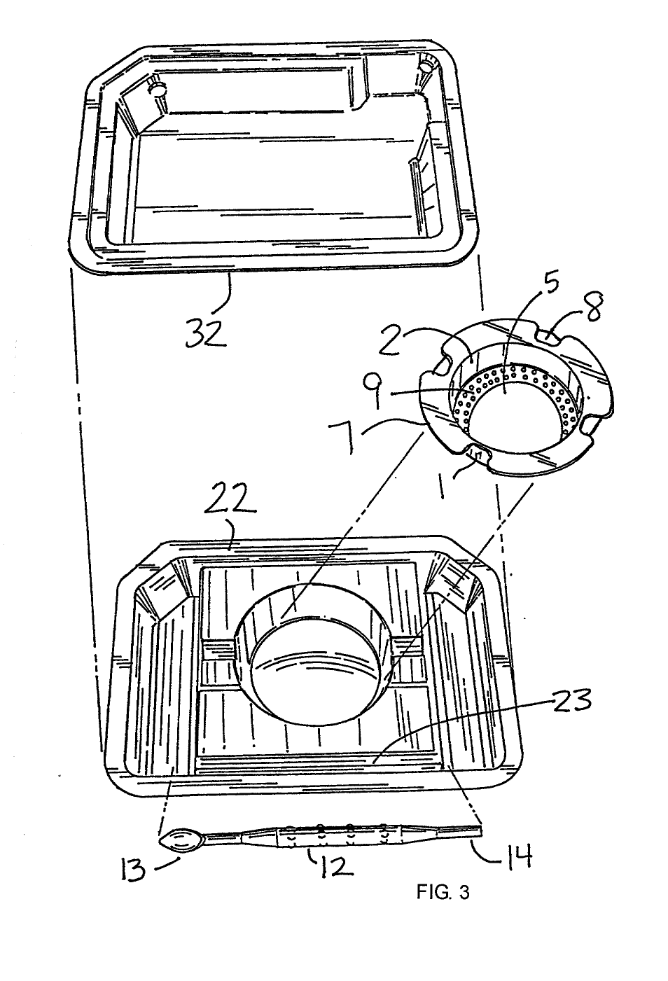

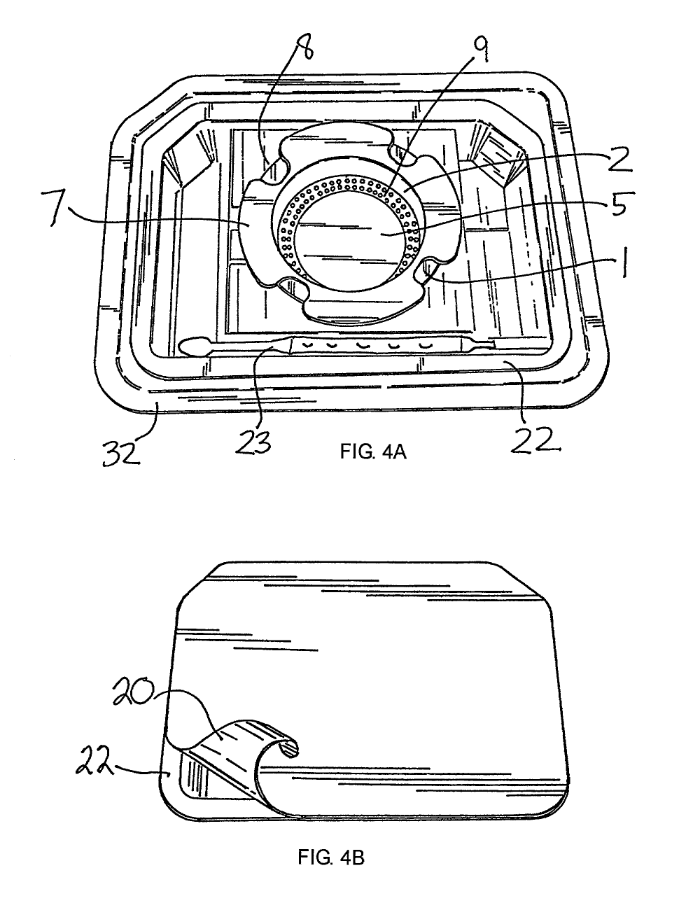

The term “cover” as used herein is defined as an entity which is placed over the bowl. The term “top” may be used interchangeably with “cover.” Representative, but not limiting, embodiments of the cover include 2 of FIG. 1, FIG. 2, FIG. 3 and FIG. 4A.

The term “double-barreled syringe” as used herein is defined as a syringe having one barrel for the particles and another barrel for a suspension material, including blood, wherein once the plunger is pushed the two products would self-mix into another barrel before exiting the syringe.

The term “finger tabs” as used herein refers to places along a rim of the cover of the bowl which facilitate placement of fingers for ease of removal of the cover from the bowl. Representative, but not limiting, embodiments of the finger tabs include 8 of FIG. 1, FIG. 2, FIG. 3 and FIG. 4A.

The term “health care provider” as used herein is defined as an individual who provides medical attention to a patient. The provider in a preferred embodiment is a surgeon, such as an orthopedic surgeon.

The term “identifying label” as used herein is an apparatus which is on or affixed to the cover of the bowl. In a preferred embodiment, the label contains informative text regarding the contents of the bowl. In a specific embodiment, the label is affixed to the cover of the bowl by adhesive. Representative, but not limiting, embodiments include 5 of FIG. 2, FIG. 3 and FIG. 4A.

The term “inner tray” as used herein is defined as a tray for housing a bowl and particles and, optionally, a mixing and delivery tool and/or a syringe containing a suspension material, such as a gel, wherein the inner tray is preferably housed inside an outer tray. It is understood that the term “inner tray” refers to an enclosure which fully encloses, for example, a bowl and particles. For example, in embodiments where the “inner tray” comprises a lid, such as 20 of FIG. 4B, the lid is a part of the inner tray. For example, where the inner tray is coated with a water-impermeable layer, it forms an enclosure around, for example, the bowl and particles, such that the contents of the inner tray (e.g., the bowl and particles) remain dry. Representative, but not limiting, embodiments of the inner tray include 22 of FIG. 3, FIG. 4A and FIG. 4B.

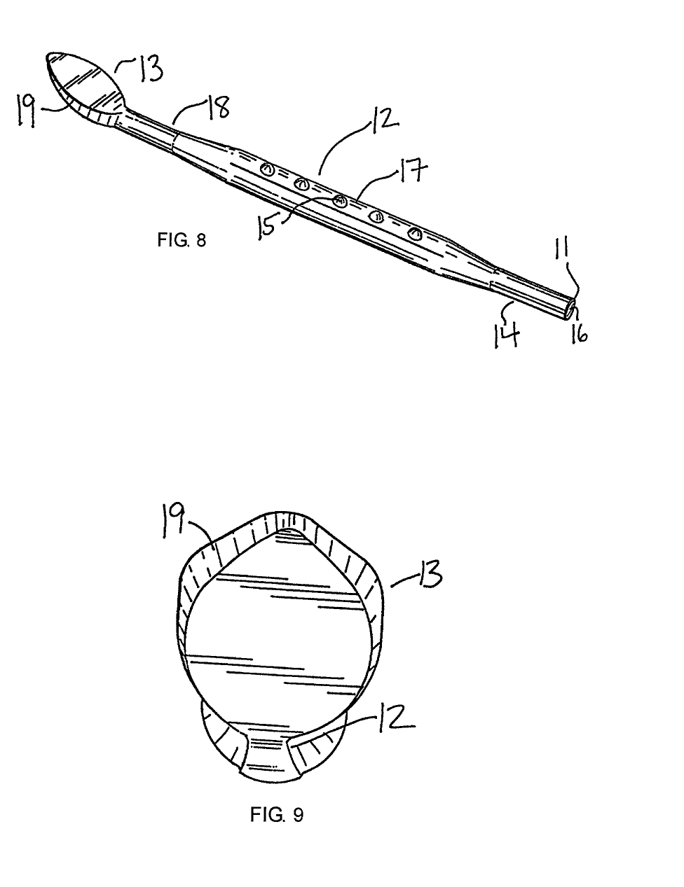

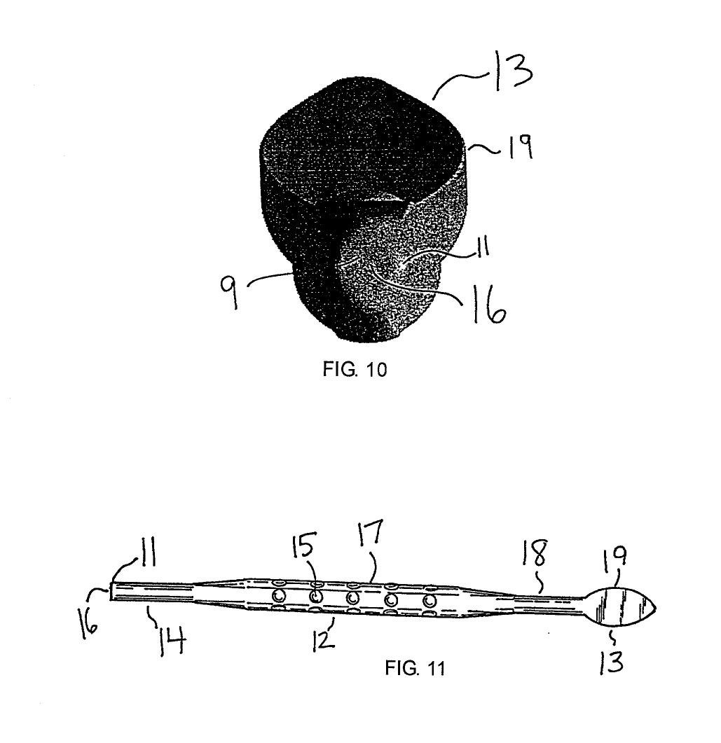

The term “mixing and delivery tool” as used herein refers to an apparatus for mixing, delivery and tamping of a particle or particles into a bone defect site. Representative, but not limiting, embodiments of the tool include 12 of FIG. 3, FIG. 4A, FIG. 6, FIG. 7, FIG. 8, FIG. 9, FIG. 10 and FIG. 11.

The term “outer tray” as used herein is defined as a tray for housing an inner tray. Representative, but not limiting, embodiments of the outer tray include 32 of FIG. 3 and FIG. 4A.

The term “peel pouch” as used herein is defined as a container in which a portion of the container is peeled away from the container upon opening of the container. In a specific embodiment, a tab is used to facilitate the peeling action. In another specific embodiment, a region, such as a comer, of the portion of the peel pouch which is peeled away lacks adhesive to facilitate peeling.

The term “plurality” as used herein is defined as more than one.

The term “rim” as used herein refers to the outer edge of the cover of the bowl. Representative, but not limiting, embodiments of the rim include 7 of FIG. 1, FIG. 2, FIG. 3 and FIG. 4A.

The term “sachet” as used herein is defined as a container such as a packet which holds a gelatinous material such as a suspension material. In a specific embodiment, the sachet is similar to a condiment packet utilized by the restaurant industry.

The term “snap-holding” as used herein is defined as the holding in place of an apparatus which is introduced into the place by a snapping motion or action.

The term “snap-holding space” as used herein is defined as the place, such as in an inner tray, for the holding of an apparatus, such as a mixing and delivery tool, which retains the apparatus following placement of the apparatus into the space by a snapping motion or action. Representative, but not limiting, embodiments of the snap-holding space include 23 of FIG. 3 and FIG. 4A. The snap-holding space may also hold a syringe, such as one containing a suspension material, such as a gel, for the particles.

The term “snap-on” as used herein refers to a cover for a bowl which is removed from or placed onto the bowl by a snapping action.

The term “sterile” as used herein is defined as being substantially free of pathogens. In a specific embodiment, sterile refers to being completely free of pathogens.

The term “surface irregularities” as used herein is defined as at least one irregularity on a surface, such as a bump, groove, channel, furrow, rut, ridge, point, crest, extension, and the like. Representative, but not limiting, embodiments of the surface irregularities include 6 of FIG. 1 or 15 of FIG. 6, FIG. 7, FIG. 8, and FIG. 11.

The term “suspension material” as used herein is defined as a material such as a gel, goo, or biological fluid for the suspension of the particles. The suspension material may be housed within the delivery system of the present invention in any means known in the art. In a specific embodiment, the suspension material is housed in a non-glass containing apparatus. In another embodiment, the suspension material is housed in a glass-containing apparatus. A skilled artisan recognizes the distinct circumstances wherein a suspension material would preferably be housed in a glass apparatus versus a non-glass apparatus. Examples of apparatuses include a syringe, a bottle, or a tube.

The term “syringe” as used herein is defined as an apparatus for injection or delivery of at least one particle into a body site, such as a bone defect. In a specific embodiment, the apparatus consists of a barrel with a plunger, wherein one end of the barrel is for the entry and movement of the plunger and the other end of the barrel is for the exiting and delivery of the particles. In a specific embodiment, the syringe is comprised of glass.

The term “tamping rod,” which may also be referred to as a tamp, as used herein is defined as an apparatus for packing or consolidating the particles, such as into a bone defect. Representative, but not limiting, embodiments of the tamping rod include 14 of FIG. 3, FIG. 6, FIG. 7, FIG. 8, and FIG. 11.

The term “trowel” as used herein is defined as an apparatus having an implement for holding, transfer and/or delivery of the particles. In a specific embodiment, the surface of the implement which holds the particles is curved. Representative, but not limiting, embodiments of the trowel include 13 of FIG. 3, FIG. 6, FIG. 7, FIG. 8, FIG. 9, FIG. 10, and FIG. 11.

The term “twist-removable” as used herein refers to a cover which is removed from or placed onto the bowl by a twisting action.

II. The Present Invention

The problem in the art which the present invention addresses regards development of a packaging and delivery system for a bone graft particle which is both sterile and glass-free and which also preferably allows a health care provider the ability to easily and safely prepare and deliver the bone graft product to a patient. In a preferred embodiment, the bone graft particle is part of a bone graft system, such as the JAX® and/or JAX® Plus Calcigel products (SMITH+NEPHEW®; Memphis, Tenn.). In an additional embodiment, the present invention also minimizes the amount of packaging. Preferably, the delivery system is designed to minimize surgical complications, such as spilling the product into the defect site. Spillage of bone graft particles, particularly those made of ceramic, into a defect site makes for difficult retrieval of the product due to undesirable adherence to soft tissue surrounding or near the defect. The present invention, therefore, is an improvement over the art because it comprises a moisture-impermeable coating for an inner tray, as opposed to the application of glass as a packaging means, to provide a moisture-free environment.

Bone graft material often times is mixed with blood or other products in an operating room or treatment room, and therefore a delivery system for a bone graft system is required that will allow a health care provider to easily mix the ceramic and suspension material of a grafting system. In addition, the packaging and delivery system of the present invention can be used to add blood or other items of a health care provider's choice to the bone graft. Once the bone graft particles are mixed with an appropriate suspension material, the present invention facilitates delivery of the particle/suspension material mixture to the bone defect, including packing the mixture into the defect. The delivery system for the bone graft substitute has been incorporated into the packaging in the present invention, and this is a considerable advantage. That is, the bone graft product is preferably packaged in a bowl with a mixing and delivery tool and is presented in a sterile manner to the health care provider without having to transfer the product to the bowl for mixing and then to the defect site in the body. Another important advantage of the present invention is the lack of a glass article to protect the moisture-sensitive bone graft particles from exposure to liquid.

In a preferred embodiment, the delivery system/packaging consists of a PETG bowl that holds the moisture-sensitive bone graft product. The bowl is placed in a PETG tray with an chlorotrifluoroethylene coating, such as an ACLAR® coating for a moisture barrier. In a specific embodiment, the ACLAR®-coated PETG tray will contain a mixing and delivery tool that the health care provider can use to mix the bone graft particles in the bowl with another product, such as a suspension material, transfer the mixture to the defect site, and tamp the mixture into the defect site. The tool preferably snaps into a long slot on the ACLAR® tray so that it will remain firmly in place during handling and shipping.

The ACLAR®-coated PETG inner tray is preferably placed in another PETG tray which has a spunbonded high-density polyethylene (HDPE), such as a TYVEK® lid. This allows the ACLAR® PETG tray to be presented to a health care provider in a sterile condition. The final packaging is placed inside a carton. Thus, the bowl and plastic instrument in the ACLAR® PETG tray are presented to a health care provider in a sterile condition, or can be kept in the ACLAR®-coated tray if the health care provider so chooses. In a specific embodiment, the package is ultimately sterilized by means well known in the art, such as by gamma irradiation.

Alternate embodiments of the delivery system include the placement of the bone graft product in a syringe or a self-mixing double barrel syringe. For a double barrel syringe, a bone graft product is placed in one barrel and another product or blood is placed in a second barrel. Once the plunger is pushed the two products would self-mix into another barrel before exiting the syringe. The bowls or instruments could come in a variety of shapes and sizes, such as a peel pouch or a series of trays and peel pouches instead of two PETG trays as described herein. Reusable instruments alternatively are used instead of the disposable one presented in the preferred embodiment. Alternate plastic instruments could come in many different shapes. One example comprises a scoop or spoon on one end and a tamp on the other.

Overall, the advantages of the packaging and delivery system of the present invention over others in the art include: 1) the primary package (bowl) becomes part of the mixing system; 2) the lid with the bumps acts as a cushion for the bone graft particles, eliminating excessive movement of the product during shipping and distribution; 3) the secondary package (Inner Tray with the foil, polyethylene, and paper lid) provides the moisture barrier, thereby eliminating the use of a glass vial, which has been an industry standard for packaging bone graft substances; and 4) the tertiary package (Outer Tray with a TYVEK® Lid) provides a double sterile barrier and facilitates introduction of the JAX® product into a sterile field.

III. Detailed Description of the Figures

FIG. 1 illustrates an embodiment of bowl 1 and a bottom view of bowl cover 2. The rim 7 of the bowl cover 2 has multiple finger tabs 8. The bottom of a recessed surface 9 of the cover 2 contains a plurality of surface irregularities 6.

FIG. 2 illustrates an embodiment of bowl 1 and a top view of bowl cover 2. The rim 7 of the bowl cover 2 has multiple finger tabs 8. The cover 2 has a rim 7 having multiple finger tabs 8, and the cover 2 also has a recessed surface 9. The recessed surface 9 has an identifying label 5 for the bowl 1 content.

FIG. 3 illustrates an embodiment of outer tray 32, inner tray 22, bowl 1, bowl cover 2 and mixing and delivery tool 12. The inner tray 22 has a snap-holding space 23. The bowl 1 has a cover 2 having a rim 7 which contains multiple finger tabs 8. The cover 2 also has a recessed surface 9, which has an identifying label 5 for the bowl 1 content. The mixing and delivery tool 12 has one end with a trowel 13 and another end with a tamping rod 14.

FIG. 4A illustrates an embodiment of an inner tray 22 housed within an outer tray 32, wherein the inner tray 22 contains both a bowl 1 having a cover 2 and a mixing and delivery tool 12. The inner tray 22 contains a snap-holding space 23 for the tool 12. The cover 2 has a rim 7 with multiple finger tabs 8, and the cover also has a recessed surface 9 with an identifying label 5.

FIG. 4B illustrates an embodiment of an inner tray 22 having a foil lid 20, which is shown herein in a partially peeled back position.

FIG. 5 illustrates an embodiment of a bowl 1 having a plurality of bone graft particles 24, wherein the bowl has a thread 25 for placement of a bowl cover.

FIG. 6 illustrates an embodiment of a mixing and delivery tool 12 having one end a trowel 13 and the other end a tamping rod 14. The trowel 13 has a curved perimeter 19. The tool 12 has a gripping region 17 which adjoins a neck region 18 connecting trowel end 13. The gripping region 17 contains multiple surface irregularities 15. The gripping region 17 in a preferred embodiment has a greater width than the neck region 18 and tamping rod 14. In a specific embodiment, the width of the neck region 18 and the tamping rod 14 are substantially similar. The tamping rod 14 end of the tool 12 has a surface 16 with a curved edge 11.

FIG. 7 illustrates an embodiment of a mixing and delivery tool 12 having one end a trowel 13 and the other end a tamping rod 14. The tool 12 contains multiple surface irregularities 15 on the gripping region 17. The neck region 18 connects the gripping region 17 with the trowel end 13. The tamping rod 14 end of the tool 12 has a surface 16 with a curved edge 11. In a preferred embodiment, the width of the gripping region 17 is greater than the width of the neck region 18 and the width of the tamping rod 14. In a specific embodiment, the width of the neck region 18 and the width of the tamping rod 14 are substantially similar.

FIG. 8 illustrates an embodiment of a mixing and delivery tool 12 having one end a trowel 13 and the other end a tamping rod 14. The tool 12 contains multiple surface irregularities 15 on the gripping region 17. The neck region 18 connects the trowel end 13 with the gripping region 17. The trowel end 13 has a curved perimeter 19. The tamping rod 14 end of the tool 12 has a surface 16 with a curved edge 11. In a preferred embodiment, the width of the gripping region 17 is greater than the width of the neck region 18 and the width of the tamping rod 14. In a specific embodiment, the width of the neck region 18 and the width of the tamping rod 14 are substantially similar.

FIG. 9 illustrates an embodiment of a mixing and delivery tool 12 having one end a trowel 13 with a curved perimeter 19.

FIG. 10 illustrates an embodiment of a mixing and delivery tool 12 having one end a trowel 13, wherein the trowel 13 has a curved perimeter 19, and the other end having a surface 16. The surface 16 has a curved edge 11 and a flat inner region 9.

FIG. 11 illustrates an embodiment of a mixing and delivery tool 12 having one end a trowel 13 and the other end a tamping rod 14 having an end surface 16. The surface 16 has a curved edge 11. The tool 12 contains multiple surface irregularities 15 in a gripping region 17. A neck region 18 connects the gripping region 17 with the trowel end 13, which has a curved perimeter 19. In a preferred embodiment, the width of the gripping region 17 is greater than the width of the neck region 18 and the width of the tamping rod 14. In a specific embodiment, the width of the neck region 18 and the width of the tamping rod 14 are substantially similar.

IV. Specific Embodiments

A. Inner Tray

The inner tray is a tray for housing a bowl, and/or a mixing and delivery tool, and/or a syringe containing a suspension material, such as a gel, wherein the inner tray is preferably housed inside an outer tray. The inner tray is moisture free, in a preferred embodiment. Furthermore, the inner tray comprises finger tabs on either side of the bowl to allow fingers to easily grasp the bowl and remove it from the tray. In a preferred embodiment, the inner tray is comprised of PETG; its polymer name is KODAR PETG 6763 Copolyester, and it is clear with blue tint. Alternate polymers include polyethylene terephthalate ester (PETE), which is a clear polyethylene plastic.

In one embodiment, the mixing and delivery tool and/or the syringe are held in the inner tray in a snap-holding space. Alternatively, the tool and/or syringe are held in an insert to the tray, such as one made of foam or any other cushioning material known to those of skill in the art.

In a preferred embodiment, the inner tray has both an anti-moisture coating, such as a chlorotrifluoroethylene coating (for instance, ACLAR®), and a lid comprising foil. In an alternative embodiment, the PETG inner tray is covered with a TYVEK® lid. The entire inner tray may be covered with the anti-moisture coating in part or in full, in alternative embodiments. In a preferred embodiment, the ACLAR® PETG tray has a lid made with foil, polyethylene, and a paper laminate which provides a moisture barrier. In a specific embodiment the lid, such as one comprising foil, is sealed to the PETG by methods standard in the art, such as with heat or adhesive.

The inner tray is preferably coated with a chlorotrifluoroethylene, such as ACLAR®. Aclar UltRx 3000 is a 3.0 millimeter chlorotrifluoroethylene (CTFE) homopolymer high performance barrier film commonly used in pharmaceutical and medical markets. (Reference Drug Master File #3764 and Canadian Drug Master File PR-MI 8933.) In an alternative embodiment, a pouch comprised of polyethylene/polyester (Poly-MYLAR®) Film is used instead of the inner tray and has a covering or side comprised of a spunbonded high-density polyethylene (HDPE), such as TYVEK®, heat-sealed to it. (both of E. I. du Pont de Nemours and Company, Inc.; Wilmington, Del.). Alternate polymers other than high-density polyethylene are known in the art.

What is important is that, in a preferred embodiment, the inner tray be coated with a layer that prevents moisture from crossing the barrier found in the inner tray. In this manner, the bone graft particles remain dry and sterile.

In another embodiment, a second inner tray comprised of PETG and having a TYVEK® lid also comprises a syringe, wherein the second inner tray further comprises a snap-holding space for the syringe or a foam insert for the syringe. A skilled artisan recognizes that the configuration wherein a second inner tray housing the syringe is separate from a first inner tray housing the particles is beneficial, given the different sterilization techniques required for each. In another embodiment, the glass syringe lies in a foam insert within an outer tray, wherein the outer tray is comprised of PETG and a TYVEK® lid.

B. Outer Tray

The outer tray contains the inner tray for the purpose of keeping the inner tray sterile. The outer tray preferably is comprised of PETG and has a spunbonded high-density polyethylene (HDPE) lid, such as a TYVEK® lid.

C. Bowl

The delivery and packaging system consists of a PETG bowl that holds the ceramic bone graft product. The bowl preferably has a twist top made with several bumps to decrease the movement of the bone graft product during shipping. Thus, the bowl is a container for storage and/or mixing of particles. In alternative embodiments, the bowl may be a basin or cup.

The cover for the bowl is an entity which is placed over the bowl. The cover may be also considered to be a lid or top of the bowl. In specific embodiments, the cover is a twist-removable cover, a snap-on cover, or a combination thereof. In a preferred embodiment, the cover comprises finger tabs. The finger tabs are places along a rim of the cover of the bowl which facilitate placement of fingers for ease of removal of the cover from the bowl. The finger tabs in a preferred embodiment are recessions which a finger fits into. In an alternative embodiment, the finger tabs are extensions to which fingers may place pressure against to facilitate removal or placement of the lid. The lid could connect to the bowl by just exerting downward pressure to pop it in the grooves.

In a preferred embodiment, the actual volume of the bowl may be at least 5 times greater than the quantity of bone graft particles, which leaves ample room in the bowl for the health care provider to mix additional products or blood into the bowl with the bone graft particles. In a specific embodiment, the volume of the quantity of the bone graft particles to the volume of the quantity of the bowl is approximately 1:20. The bowl also has a low profile to minimize the package size while still fitting into the palm of a hand for easy mixing. In a specific embodiment, the bowl of the present invention is a single-sized bowl. Different sized bowls containing, for instance 5, 10, or 20 cc quantities of bone graft particles, are another embodiment of the present invention. In an alternative embodiment of the present invention, separate sized bowls for each of the different quantities of bone graft particles are utilized.

D. Mixing and Delivery Tool

The mixing and delivery tool is an apparatus for mixing, delivery and tamping of a bone graft particle or particles. In other embodiments, the tool may be an implementation, device, means, utensil or instrument. In preferred embodiments, it is a disposable plastic stirring/tamping rod that a health care provider can use to mix the bone graft particles in the bowl with another product, such as a suspension material, transfer the mixture to the defect site, and tamp the mixture into the defect site.

The tool preferably consists of a trowel on one end and a tamping rod on the other. The instrument also has a bump and groove design along the length to allow easier handling and a more tactile response with gloved hands during surgery. The tamp end of the instrument facilitates compression of the product into small defects, while the trowel end allows delivery of the product to small defects (the tip of the trowel) and large defect sites (the entirety of the trowel). Because of the trowels' thin design, finite element analysis was used to maintain strength while providing a low profile instrument. Finite element analysis is a computerized stress analysis in the interest of optimizing the design of a tool commonly known in the art. The analysis facilitates design of a tool regarding loading of various forces. Computer software programs for such analysis are known in the art, such as Pro/MECHANICA® (PARAMETRIC TECHNOLOGY CORPORATION®, Waltham, Mass.).

The trowel end of the tool has an implement for holding, transfer and/or delivery of the particles. In a specific embodiment, the surface of the implement which holds the particles is curved which provides an important advantage over others in the art to prevent spillage of bone graft particles or a bone graft particle/suspension material mixture. In alternative embodiments, the trowel end is a scoop or spoon. The tamping rod, which may also be referred to as a tamp, is an apparatus for packing or consolidating the particles, such as into a bone defect.

In a preferred embodiment the tool is comprised of plastic material, such as polycarbonate, polystyrene, polypropylene, cellulose, polyphenylene oxide, and polyphenylene sulfide and the like.

All of the systems and compositions disclosed and claimed herein can be made and executed without undue experimentation in light of the present disclosure. While the systems and compositions of this invention have been described in terms of preferred embodiments, it will be apparent to those of skill in the art that variations may be applied to the systems and compositions without departing from the concept, spirit, and scope of the invention. More specifically, it will be apparent that certain agents which are both chemically, structurally and physiologically related may be substituted for the agents described herein while the same or similar results would be achieved. All such similar substitutes and modifications apparent to those skilled in the art are deemed to be within the spirit, scope, and concept of the invention as defined by the appended claims.

We claim:

1. A delivery system for a plurality of bone graft particles, comprising:
a. a bowl;
b. an inner tray for housing said bowl;
c. an outer tray for housing said inner tray; and
d. said plurality of bone graft particles.

2. The delivery system of claim 1, further comprising a mixing and delivery tool.

3. The delivery system of claim 1, further comprising a sterile suspension material.

4. The delivery system of claim 3, wherein said suspension material is housed in a container selected from the group consisting of a syringe, sachet, Applipak®, and peel pouch.

5. The delivery system of claim 3, wherein said suspension material is housed in a syringe.

6. The delivery system of claim 1, wherein said bowl contains said plurality of bone graft particles.

7. The delivery system of claim 1, wherein the bowl and plurality of bone graft particles are housed within the inner tray.

8. The delivery system of claim 7, wherein said inner tray is impermeable to water.

9. The delivery system of claim 1, wherein said bowl further comprises a cover selected from the group consisting of a twist-removable cover, a snap-on cover, or a combination thereof.

10. The delivery system of claim 9, wherein a surface of said cover further comprises a plurality of surface irregularities.

11. The delivery system of claim 10, wherein said surface irregularities are selected from the group consisting of bumps, bulges, grooves, channels, furrows, ruts, points, crests and ridges.

12. The delivery system of claim 10, wherein said surface irregularities are bumps.

13. The delivery system of claim 6, wherein the ratio of the volume of said particles to the volume of said bowl is at least approximately 1:5.

14. The delivery system of claim 6, wherein the ratio of the volume of said particles to the volume of said bowl is approximately 1:20.

15. The delivery system of claim 9, wherein said cover comprises a recessed surface, wherein said surface is on a plane parallel with a topmost horizontal plane of said bowl, and wherein when said cover is on said bowl, said plane of said recessed surface lies beneath said topmost horizontal plane of said bowl.

16. The delivery system of claim 9, wherein said cover comprises a plurality of finger tabs along a rim of said cover.

17. The delivery system of claim 2, wherein said delivery and mixing tool comprises a first end having a trowel and a second end having a tamping rod.

18. The delivery system of claim 2, wherein said tool further comprises a surface having a plurality of surface irregularities.

19. The delivery system of claim 18, wherein said surface irregularities are selected from the group consisting of bumps, bulges, grooves, channels, furrows, ruts, points, crests, and ridges.

20. The delivery system of claim 2, wherein said tool is a plastic material.

21. The delivery system of claim 20, wherein said plastic material is selected from the group consisting of polycarbonate, polystyrene, polypropylene, cellulose, polyphenylene oxide, and polyphenylene sulfide.

22. The delivery system of claim 1, wherein said inner tray is polyethylene terephthalate glycol (PETG).

23. The delivery system of claim 1, wherein a surface of said inner tray comprises a coating of chlorotrifluoroethylene.

24. The delivery system of claim 1, wherein a surface of said inner tray comprises an ACLAR® coating.

25. The delivery system of claim 2, wherein said inner tray further comprises a snap-holding space for said tool.

26. The delivery system of claim 1, wherein said inner tray comprises a lid, wherein said lid comprises a layer selected from the group consisting of foil, polyethylene, paper laminate and a combination thereof.

27. The delivery system of claim 1, wherein said inner tray comprises a lid, wherein said lid comprises a foil layer, a polyethylene layer, and a paper laminate layer.

28. The delivery system of claim 1, wherein said inner tray comprises a chlorotrifluoroethylene coating and a foil lid.

29. The delivery system of claim 1, wherein said inner tray comprises an ACLAR® coating and a foil lid.

30. The delivery system of claim 1, wherein said outer tray is comprised of polyethylene terephthalate glycol (PETG).

31. The delivery system of claim 1, wherein said outer tray further comprises a lid, wherein said lid is comprised of high-density polyethylene.

32. The delivery system of claim 1, wherein said outer tray further comprises a lid, wherein said lid is made of TYVEK®.

33. The delivery system of claim 1, wherein said bowl, said particles, said inner tray and said outer tray are housed in a container.

34. The delivery system of claim 33, wherein said container is selected from the group consisting of a carton, a bag, a box, a pack, a pocket, a peel pouch, a series of trays, and a combination thereof.

35. The delivery system of claim 2, wherein said bowl, said particles, said tool, said inner tray and said outer tray are housed in a container.

36. The delivery system of claim 35, wherein said container is selected from the group consisting of a carton, a bag, a box, a pack, a pocket, a peel pouch, a series of trays, and a combination thereof.

37. The delivery system of claim 3, wherein said bowl, said particles, said suspension material, said outer tray, and said inner tray are housed in a container.

38. The delivery system of claim 37, wherein said container is selected from the group consisting of a carton, a bag, a box, a pack, a pocket, a peel pouch, a series of trays, and a combination thereof.

39. The delivery system of claim 1, wherein said bowl, said plurality of particles, said tool, and said inner tray are sterile.

40. A delivery system for a plurality of particles, comprising:
a. a bowl containing a plurality of particles;
b. a syringe comprising a sterile suspension material;
c. an inner tray for housing said bowl and said tool; and
d. an outer tray for housing said inner tray.

41. The delivery system of claim 5, wherein said syringe is a double-barreled syringe.

42. A packaging system for sterile bone graft particles comprising:
a. an inner tray;
b. a plurality of bone graft particles housed within said inner tray, wherein said tray is impermeable to water such that said bone graft particles remain free of water; and
c. an outer tray for housing said inner tray.

43. The packaging system of claim 42, further comprising a mixing and delivery tool.

44. The packaging system of claim 42, further comprising a bowl.

45. The packaging system of claim 42, further comprising a sterile suspension material.

46. The packaging system of claim 45, wherein said suspension material is housed in a container selected from the group consisting of a syringe, sachet, Applipak®, and peel pouch.

47. The packaging system of claim 45, wherein said suspension material is housed in a syringe.

48. The packaging system of claim 44, wherein said bowl contains said plurality of bone graft particles.

49. The packaging system of claim 48, wherein the bowl and the plurality of bone graft particles are housed within the inner tray.

50. The packaging system of claim 42, wherein the inner tray is impermeable to water.

51. The packaging system of claim of claim 44, wherein said bowl further comprises a cover selected from the group consisting of a twist-removable cover, a snap-on cover, or a combination thereof.

52. The packaging system of claim 51, wherein a surface of said cover further comprises a plurality of surface irregularities.

53. The packaging system of claim 52, wherein said surface irregularities are selected from the group consisting of bumps, bulges, grooves, channels, furrows, ruts, points, crests and ridges.

54. The packaging system of claim 52, wherein said surface irregularities are bumps.

55. The packaging system of claim 48, wherein the ratio of the volume of said particles to the volume of said bowl is at least approximately 1:5.

56. The packaging system of claim 48, wherein the ratio of the volume of said particles to the volume of said bowl is approximately 1:20.

57. The packaging system of claim 51, wherein said cover comprises a recessed surface, wherein said surface is on a plane parallel with a topmost horizontal plane of said bowl, and wherein when said cover is on said bowl, said plane of said recessed surface lies beneath said topmost horizontal plane of said bowl.

58. The packaging system of claim 51, wherein said cover comprises a plurality of finger tabs along a rim of said cover.

59. The packaging system of claim 43, wherein said delivery and mixing tool comprises a first end having a trowel and a second end having a tamping rod.

60. The packaging system of claim 43, wherein said tool further comprises a surface having a plurality of surface irregularities.

61. The packaging system of claim 60, wherein said surface irregularities are selected from the group consisting of bumps, bulges, grooves, channels, furrows, ruts, points, crests and ridges.

62. The packaging system of claim 43, wherein said tool is a plastic material.

63. The packaging system of claim 62, wherein said plastic material is selected from the group consisting of polycarbonate, polystyrene, polypropylene, cellulose, polyphenylene oxide and polyphenylene sulfide.

64. The packaging system of claim 42, wherein said inner tray is polyethylene terephthalate glycol (PETG).

65. The packaging system of claim 42, wherein a surface of said inner tray comprises a chlorofluorotriethylene coating.

66. The packaging system of claim 42, wherein a surface of said inner tray comprises an ACLAR® coating.

67. The packaging system of claim 42, wherein said inner tray further comprises a snap-holding space for said tool.

68. The packaging system of claim 42, wherein said inner tray comprises a lid, wherein said lid comprises a layer selected from the group consisting of foil, polyethylene, paper laminate and a combination thereof.

69. The packaging system of claim 42, wherein said inner tray comprises a lid, wherein said lid comprises a foil layer, a polyethylene layer, and a paper laminate layer.

70. The packaging system of claim 42, wherein said inner tray comprises a chlorotrifluoroethylene coating and a foil lid.

71. The packaging system of claim 42, wherein said inner tray comprises an ACLAR® coating and a foil lid.

72. The packaging system of claim 42, wherein said outer tray is polyethylene terephthalate glycol (PETG).

73. The packaging system of claim 42, wherein said outer tray further comprises a lid, wherein said lid is high-density polyethylene.

74. The packaging system of claim 42, wherein said outer tray further comprises a lid, wherein said lid is made of TYVEK®.

75. The packaging system of claim 44, wherein said bowl, said particles, said inner tray and said outer tray are housed in a container.

76. The packaging system of claim 75, wherein said container is selected from the group consisting of a carton, a bag, a box, a pack, a pocket, a peel pouch, a series of trays, and a combination thereof.

77. The packaging system of claim 42, further comprising a bowl and a mixing and delivery tool, wherein said bowl, said particles, said tool, said inner tray and said outer tray are housed in a container.

78. The packaging system of claim 77, wherein said container is selected from the group consisting of a carton, a bag, a box, a pack, a pocket, a peel pouch, a series of trays, and a combination thereof.

79. The packaging system of claim 42, further comprising a bowl, a mixing and delivery tool, and a suspension material, wherein said bowl, said particles, said tool, said suspension material, said inner tray and said outer tray are housed in a container.

80. The packaging system of claim 79, wherein said container is selected from the group consisting of a carton, a bag, a box, a pack, a pocket, a peel pouch, a series of trays, and a combination thereof.

81. The packaging system of claim 44, wherein said bowl, said plurality of particles, said tool and said inner tray are sterile.

82. The delivery system of claim 5, wherein said syringe is housed in a snap-holding space of said inner tray.

83. The delivery system of claim 5, wherein said syringe is housed in an insert of said inner tray.

84. The delivery system of claim 83, wherein said insert is comprised of foam.

85. The delivery system of claim 5, wherein said syringe is housed in a snap-holding space in a second inner tray.

86. The delivery system of claim 5, wherein said syringe is housed in an insert of said outer tray.

87. The delivery system of claim 86, wherein said insert is comprised of foam.
